(19) United States
(12) Patent Application Publication
(10) Pub. No.: US 2005/0163849 A1
(43) Pub. Date: Jul. 28, 2005

(54) Compositions and dosage forms for enhanced absorption of iron

(76) Inventors: Patrick S.L. Wong, Burlingame, CA (US); Dong Yan, Cupertino, CA (US); George Guittard, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,137

(22) Filed: Oct. 29, 2004

Related U.S. Application Data
(60) Provisional application No. 60/516,259, filed on Oct. 31, 2003.
(60) Provisional application No. 60/519,509, filed on Nov. 12, 2003.

(51) Int. Cl. A61K 31/295; A61K 9/24

(52) U.S. Cl. 424/473; 514/502

(57) ABSTRACT

A complex comprised of iron and a transport moiety, such as a fatty acid, is described. The complex has an enhanced absorption in the gastrointestinal tract, particularly the lower gastrointestinal tract. The complex, and compositions and dosage forms prepared using the complex, provide for absorption by the body of iron through a period of ten to twenty-four hours, thus enabling a true once-daily dosage form for iron.

### CROSS-REFERENCE TO RELATED APPLICATIONS

This application claims the benefit of U.S. provisional patent application No. 60/516,259, filed Oct. 31, 2003, and of U.S. provisional patent application No. 60/519,509, filed Nov. 12, 2003, both applications are incorporated herein by reference in their entirety.

### FIELD OF THE INVENTION

This invention relates to the compositions and dosage forms for delivery of iron. More particularly, the invention relates to a complex of iron and a transport moiety where the complex provides an enhanced absorption of iron in the gastrointestinal tract, and more particularly, in the colon.

### BACKGROUND OF THE INVENTION

Elemental iron and a variety of iron compounds have been conventionally used as hematinics in the therapeutic treatment of anemias and as nutritional supplements to insure satisfaction of the bodies minimum daily recommended allowance of iron. Typically, iron is administered in combination with other minerals and/or vitamins for which minimum recommended daily allowances have been established. Iron supplements generally include a single form of iron, for example, an iron (II) salt (i.e. a salt containing divalent or ferrous iron), an iron (III) salt (i.e. a salt containing trivalent or ferric iron), or iron (O) powder (e.g. carbonyl iron, typically made by heating gaseous iron pentacarbonyl, Fe(CO)₅.

Many prior art iron nutritional supplements contain a rapid release dosage form of iron, which is typically an iron salt, such as ferrous sulfate, since certain iron salts are more soluble in gastrointestinal fluids than certain other salts and metallic iron forms. However, a rapid release dosage form can cause an excessively high maximum concentration of iron in the blood (Cmax) and a short Tmax, or time lapse between administration of the supplement and attainment of Cmax. The high Cmax of the prior art iron formulations can cause unpleasant, harmful, or even fatal side effects (Crosby, Arch. Intem. Med., 138: 766-767 (1978)). For example, heartburn, nausea, upper G.I. discomfort, constipation, and diarrhea are common. Side effects appear to be dose related. For example, 25% of individuals treated with a dose of 200 mg of iron per day, divided into three equal portions, reported undesirable symptoms, compared with an incidence of 13% in patients treated with placebo; undesirable side effects increased to 40% when the iron dosage was increased to 400 mg (Hillman, R. S., GOODMAN & GILMAN'S THE PHARMACOLOGICAL BASIS OF THERAPIES, Chapter 53, page 1311, Ninth Edition, McGraw Hill, 1996)). Due, at least in part to the side effects, anemia is under treated in patients.

Another problem associated with oral iron therapy is its limited bioavailability. For example, 40% of a total daily dose of 35 mg given orally is absorbed and only 18% of a 195 mg oral daily dose is absorbed (Hillman, R. S., GOODMAN & GILMAN'S THE PHARMACOLOGICAL BASIS OF THERAPIES, Chapter 53, page 1323, Ninth Edition, McGraw Hill, 1996)). The decreasing absorption with increasing dose makes it difficult to treat iron-deficiency anemia, and several small doses each day are required to maximize absorption.

Absorption of the compounds, and of iron salts, is far greater in the upper G.I. tract due to its larger surface area relative to the lower G.I. tract. The lower G.I. tract, or colon, lacks microvilli which are present in the upper G.I. tract. The presence of microvilli greatly increases the surface area for drug absorption, and the upper G.I. tract has 480 times the surface area than does the colon. Differences in the cellular characteristics of the upper and lower G.I. tracts also contribute to the poor absorption of molecules in the lower G.I. tract. FIG. 1 illustrates two common routes for transport of compounds across the epithelium of the G.I. tract. Individual epithelial cells, represented by 10a, 10b, 10c, form a cellular barrier along the small and large intestine. Individual cells are separted by water channels or tight junctions, such as junctions 12a, 12b. Transport across the epithelium occurs via either or both a transcellular pathway and a paracellular pathway. The transcellular pathway for transport, indicated in FIG. 1 by arrow 14, involves movement of the compound across the wall and body of the epithelial cell by passive diffusion or by carrier-mediated transport. The paracellular pathway of transport involves movement of molecules through the tight junctions between individual cells, as indicated by arrow 16. Paracellular transport is less specific but has a much greater overall capacity, in part because it takes place throughout the length of the G.I. tract. However, the tight junctions vary along the length of the G.I. tract, with an increasing proximal to distal gradient in effective ‘tightness’ of the tight junction. Thus, the duodenum in the upper G.I. tract is more “leaky” than the ileum in the upper G.I. tract which is more “leaky” than the colon, in the lower G.I. tract (Knauf, H. et al., Klin. Wochenschr., 60(19): 1191-1200 (1982)).

Since the typical residence time of a drug in the upper G.I. tract is from approximately four to six hours, drugs having poor colonic absorption are absorbed by the body through a period of only four to six hours after oral ingestion. Frequently it is medically desirable that the administered drug be presented in the patient's blood stream at a relatively constant concentration throughout the day. To achieve this with traditional drug formulations that exhibit minimal colonic absorption, patients would need to ingest the drugs three to four times a day. Practical experience with this inconvenience to patients suggests that this is not an optimum treatment protocol. Accordingly, it is desired that a once daily administration of such drugs, with long-term absorption throughout the day, be achieved.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only four to six hours, the fact that a proposed controlled release dosage form may release its payload after the residence period of the dosage form in the upper G.I. does not mean the that body will continue to absorb the controlled release drug past the four to six hours of upper G.I. residence. Instead, the drug released by the controlled release dosage form after the dosage form has entered the lower G.I. tract is generally not absorbed and, instead, is expelled from the body.

Thus, there remains a need to treat iron-deficiency in humans, and in particular a need to provide an iron dosage form that permits absorption of iron in both the upper and lower gastrointestinal tract, to maximize the bioavailability achieved for a given iron dose. A means to enhance absorption of iron, particularly in the lower G.I. tract, would provide a significant advancement in the art by permitting a once-daily iron treatment system with reduced side effects.

### SUMMARY OF THE INVENTION

Accordingly, in one aspect the invention provides a substance comprised of iron and a transport moiety, the iron and the transport moiety forming a complex.

In one embodiment, the transport moiety is a fatty acid of the form CH₃(CₙH₂ₙ)COOH, where n is from 4-16. In preferred embodiment, the fatty acid is capric acid or lauric acid.

In another aspect, the invention includes a composition, comprising, a complex consisting essentially of iron and a transport moiety, and a pharmaceutically acceptable vehicle, wherein the complex has an absorption in the lower gastrointestinal tract that is at least 2 fold higher than the absorption of ferrous sulfate in the lower gastrointestinal tract.

In another aspect, the invention provides a dosage form comprising the composition described above.

In another aspect, the invention provides a dosage form comprising the complex described above.

In one embodiment, the dosage form is an osmotic dosage form. The dosage form can be comprised of (i) a push layer; (ii) drug layer comprising an iron-transport moiety complex; (iii) a semipermeable wall provided around the push layer and the drug layer; and (iv) an exit. Alternatively, the dosage form can comprise (i) a semipermeable wall provided around an osmotic formulation comprising an iron-transport moiety complex, an osmagent, and an osmopolymer; and (ii) an exit.

In one embodiment, the dosage form provides a total iron daily dose of between 20-400 mg.

In another aspect, the invention provides an improvement in a dosage form comprising iron. The improvement comprises a dosage form including a complex comprised of iron and a transport moiety.

In another aspect, the invention includes a method for treating an iron-deficiency in a subject, comprising administering the composition or dosage form described above. In one embodiment, the composition or dosage form is administered orally.

In another aspect, the invention provides a method of preparing an iron-transport moiety complex. The method includes providing iron; providing a transport moiety; combining the iron and the transport moiety in the presence of a solvent having a dielectric constant less than that of water; whereby the combining results in formation of a complex between the iron and the transport moiety.

In one embodiment, the iron and the transport moiety are combined in a solvent having a dielectric constant at least two fold lower than the dielectric constant of water. Exemplary solvents include methanol, ethanol, acetone, benzene, methylene chloride, and carbon tetrachloride.

In another aspect, the invention provides a method of improving gastrointestinal absorption of iron, comprising providing a complex consisting essentially of iron and a transport moiety, and administering the complex to a patient.

In one embodiment, the improved absorption comprises improved absorption in the lower gastrointestinal tract.

In another embodiment, the improved absorption comprises improved absorption in the upper gastrointestinal tract.

In another aspect, the invention includes a substance comprising iron and a transport moiety, where the substance is prepared by a process comprising (i) providing iron; (ii) providing a transport moiety; (iii) combining the iron and the transport moiety in the presence of a solvent having a dielectric constant less than that of water, where the combining forms a complex between iron and the transport moiety.

These aspects, as well as other aspects, features, and advantages of the invention will become more apparent from the following detailed disclosure of the invention and its aspects.

### BRIEF DESCRIPTION OF THE FIGURES

The following figures are not drawn to scale, and are set forth to illustrate various embodiments of the invention.

FIG. 1 is a diagram of epithelial cells of the gastrointestinal tract, illustrating the transcellular pathway and the paracellular pathway for transport of molecules through the epithelium;

FIG. 2A shows a generalized synthetic reaction scheme for preparation of an iron-transport moiety complex;

FIG. 2B shows a generalized synthetic reaction scheme for preparation of an iron-transport moiety complex, where the transport moiety includes a carboxyl group;

FIG. 2C shows a synthetic reaction scheme for preparation of an iron-fatty acid complex;

FIG. 3 illustrates an exemplary osmotic dosage form shown in cutaway view,

FIG. 4 illustrates another exemplary osmotic dosage form for a once daily dosing of iron, the dosage form comprising an iron-fatty acid complex with an optional loading dose of the complex or of iron in the outer coating;

FIG. 5 illustrates one embodiment of a once daily iron dosage form comprising both iron and iron-fatty acid complex, with an optional loading dose of iron by coating;

FIGS. 6A-6C illustrate an embodiment of a dosage prior to administration to a subject and comprising an iron-fatty acid complex in a matrix (FIG. 6A), in operation after ingestion into the gastrointestinal tract (FIG. 6B), and after sufficient erosion of the matrix has caused separation of the banded sections of the device (FIG. 6C).

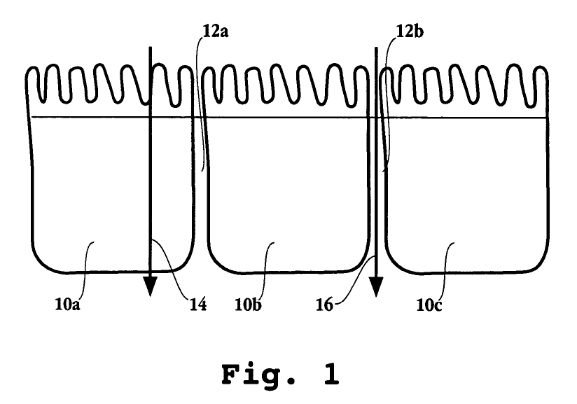

### DETAILED DESCRIPTION OF THE INVENTION

The present invention is best understood by reference to the following definitions, drawings, and exemplary disclosure provided herein.

I. Definitions

By “composition” is meant one or more of an iron-transport moeity complexe, optionally in combination with additional active pharmaceutical ingredients, and/or optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:
ΔLogD=LogD(complex)−LogD(loose-ion pair)≧0.15 (Equation 1)
wherein, D, the distribution coefficient (apparent partition coefficient), is the ratio of the equilibrium concentration of all species of the drug moiety and the transport moiety in octanol to the same species in water (deionized water) at a set pH (typically about pH=5.0 to about pH=7.0) and at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. For instance, the octanol/water apparent partition coefficient (D=C_{octanol}/C_{water}) of a putative complex (in deionized water at 25 degree Celsuis) can be determined and compared to a 1:1 (mol/mol) physical mixture of the transport moiety and the drug moiety in deionized water at 25 degree Celsuis. If the difference between the Log D for the putative complex (D+T−) and the Log D for the 1:1 (mol/mol) physical mixture, D⁺|T⁻ is determined is greater than or equal to 0.15, the putative complex is confirmed as being a complex according to the invention. In preferable embodiments, Δ Log D≧0.20, and more preferably Δ Log D≧0.25, more preferably still Δ Log D≧0.35.

By “dosage form” is meant a pharmaceutical composition in a medium, carrier, vehicle, or device suitable for administration to a patient in need thereof.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element.

By “fatty acid” is meant any of the group of organic acids of the general formula CH₃(CₙHₓ)COOH where the hydrocarbon chain is either saturated (x=2n, e.g. palmitic acid, C₁₅H₃₁COOH) or unsaturated (x=2n−2, e.g. oleic acid, CH₃C₁₆H₃₀COOH).

By “intestine” or “gastrointestinal (G.I.) tract” is meant the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

The term “iron” intends iron (Fe) in any of its oxidative states and in combination with any salt. “Ferrous” refers to iron with a +2 charge (also denoted in the art as Fe2+, Fe++, iron (II)). “Ferric” refers to iron with a +3 charge (also denoted in the art as Fe3+, Fe+++, iron (III)). Exemplary ferrous salts and ferric salts include, but are not limited to ferrous and ferric sulfate, fumarate, succinate, gluconate, etc.

By “loose ion-pair” is meant a pair of ions that are, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract” or “lower G.I. tract” is meant the large intestine.

By “patient” is meant an animal, preferably a mammal, more preferably a human, in need of therapeutic intervention.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “transport moiety” is meant a compound that is capable of forming, or a residue of that compound that has formed, a complex with a drug, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0.

By “pharmaceutical composition” is meant a composition suitable for administration to a patient in need thereof.

By “structural element” is meant a chemical group that (i) is part of a larger molecule, and (ii) possesses distinguishable chemical functionality. For example, an acidic group or a basic group on a compound is a structural element.

By “substance” is meant a chemical entity having specific characteristics.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO− being a residual structural element.

By “upper gastrointestinal tract” or “upper G. I. tract” is meant that portion of the gastrointestinal tract including the stomach and the small intestine.

II. Iron Complex Formation and Characterization

As noted above, iron deficiency is a common cause of nutritional anemia in humans. Iron is an essential component of myoglobin, heme enzymes, and metalloflavoprotein enzymes. Iron deficiency can affect metabolism in muscle independently of the effect of anemia on oxygen delivery (Hillman, R. S., GOODMAN & GILMAN'S THE PHARMACOLOGICAL BASIS OF THERAPIES, Chapter 53, page 1311, Ninth Edition, McGraw Hill, 1996)). Poor absorption of iron and the side effects resulting from increasing doses of iron make it difficult to treat iron deficiencies in a patiently friendly manner.

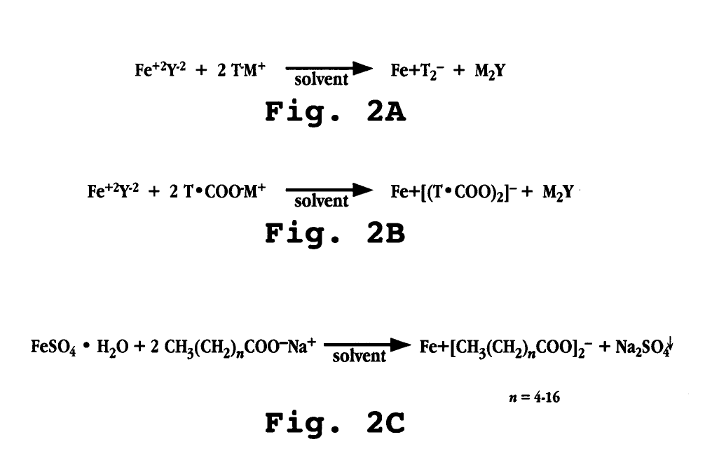

Accordingly, in one aspect, the invention provides a compound comprised of iron and a transport moiety, the two species complexed together in a manner that permits an enhanced absorption of the compound in the lower G.I. tract. The compound permits formulation of compositions and dosage forms for once-daily dosing of iron. The iron-transport moiety complex is prepared according to the general synthetic reaction scheme illustrated in FIG. 2A. Briefly, iron, in the form of an iron salt such the general ferrous salt Fe⁺²Y⁻² indicated in the drawing is combined with a transport moiety, represented as T⁻M⁺ in the drawing. Exemplary transport moieties are listed above and include fatty acids, benzenesulfonic acid, benzoic acid, fumaric acid, and salicylic acid. The two species are contacted in the presence of an organic solvent that has a dielectric constant less than water, as will be discussed below, to form an iron-transport moiety complex where the species are associated by a tight-ion pair bond, as denoted in FIG. 2A by the representation Fe+(T₂)−. The species in the complex are not covalently bound; the advantages provided by the non-covalent bonding are discussed below.

FIG. 2B illustrates a more specific synthetic reaction scheme for formation of an iron-transport moiety complex. In this scheme, the transport moiety, T⁻, is represented as a species having a carboxyl group (COO⁻). The carboxyl-containing transport moiety, T−COO⁻, is mixed in an organic solvent having a dielectric constant less than water, to form a complex of iron and the transport moiety associated by a tight-ion pair bond, denoted in the drawing as Fe+[(T−COO)₂]−.

Example 1 describes preparation of an exemplary iron-transport moiety complex, ferrous laurate. Briefly, and as illustrated in FIG. 2C, a solution of the transport moiety, such as sodium laurate, in an organic solvent is prepared. An iron-containing solution, such as ferrous sulfate, in an organic solvent is prepared. The solution containing the iron is added to the solution containing the transport moiety to form a ferrous-laurate complex, the species in the complex associated by a non-covalent bond tight-ion pair bond.

In Example 1, a complex was prepared using lauric acid as a representative transport moiety. It will be understood that lauric acid is merely exemplary and that the procedure is equally applicable to species other than fatty acids and to fatty acids of any carbon chain length. In particular, the invention contemplates complex formation of iron with various fatty acids or salts of fatty acids, the fatty acids having from 4 to 20 carbon atoms, more preferably 6 to 18 carbon atoms and even more preferably 8 to 18 carbon atoms. The fatty acids or their salts can be saturated or unsaturated. Exemplary saturated fatty acids contemplated for use in preparation of the complex include butanoic (butyric, 4C); pentanoic (valeric, 5C); hexanoic (caproic, 6C); octanoic (caprylic, 8C); nonanoic (pelargonic, 9C); decanoic (capric, 1° C.); dodecanoic (lauric, 12C); tetradecanoic (myristic, 14C); hexadecanoic (palmitic, 16C); heptadecanoic (margaric, 17C); and octadecanoic (stearic, 18C), where the systematic name is followed in parenthesis by the trivial name and the number of carbon atoms in the fatty acid. Unsaturated fatty acids include oleic acid, linoleic acid, and linolenic acid, all having 18 carbon atoms. Linoleic acid and linolenic acid are polyunsaturated.

Complex formation of iron with alkyl sulfates or a salt of an alkyl sulfate is also contemplated, where the alkyl sulfate may be saturated or unsaturated. Exemplary alkyl sulfates, or their salts (sodium potassium, magnesium, etc), have from 4 to 20 carbon atoms, more preferably 6 to 18 and even more preferably 8 to 18 carbon atoms. Complex formation of iron with the benzenesulfonic acid, benzoic acid, fumaric acid, and salicylic acid, or the salts of these acids, is also contemplated.

With continuing reference to Example 1, the complex consisting of ferrous-laurate is prepared from methanol. Methanol is merely an exemplary solvent, and other solvents in which fatty acids are soluble are suitable. For example, fatty acids are soluble in chloroform, benzene, cyclohexane, ethanol (95%), acetic acid, and acetone. The solubility (in g/L) of capric acid, lauric acid, myristic acid, palmitic acid, and stearic acid in these solvents is indicated in Table 1.

TABLE 1
Solubility (g/L) of Fatty Acids at 20° C.

| Fatty Acid | | | | | Ethanol | Acetic | | |
|---|---|---|---|---|---|---|---|---|
| (no. carbons) | Chloroform | Benzene | Cyclohexanne | Acetone | 95% | acid | Methanol | Acetonitrile |
| Capric (10) | 3260 | 3980 | 3420 | 4070 | 4400 | 5670 | 5100 | 660 |
| Lauric (12) | 830 | 936 | 680 | 605 | 912 | 818 | 1200 | 76 |
| myristic | 325 | 292 | 215 | 159 | 189 | 102 | 173 | 18 |
| (14) | | | | | | | | |
| palmitic | 151 | 73 | 65 | 53.8 | 49.3 | 21.4 | 37 | 4 |
| (16) | | | | | | | | |
| stearic (18) | 60 | 24.6 | 24 | 15.4 | 11.3 | 1.2 | 1 | <1 |

In one embodiment, the solvent used for formation of the complex is a solvent having a dielectric constant less than water, and preferably at least two fold lower than the dielectric constant of water, more preferably at least three-fold lower than that of water The dielectric constant is a measure of the polarity of a solvent and dielectric constants for exemplary solvents are shown in Table 2.

TABLE 2
Characteristics of Exemplary Solvents

| Solvent | Boiling Pt., ° C. | Dielectric constant |
|---|---|---|
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide (DMF) | 153 | 38.3 |
| dimethyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the iron-based complex. Dipolar aprotic solvents do not contain an OH bond but typically have a large bond dipole by virtue of a multiple bond between carbon and either oxygen or nitrogen. Most dipolar aprotic solvents contain a C—O double bond. The dipolar aprotic solvents noted in Table 2 have a dielectric constant at least two-fold lower than water.

While not wishing to be bound by specific understanding of mechanisms, the inventors reason as follows. When loose ion-pairs are placed in a polar solvent environment, it is assumed that polar solvent molecules will insert themseives in the space occupied by the ionic bond, thus driving apart the bound ions. A solvation shell, comprising polar solvent molecules electrostatically bonded to a free ion, may be formed around the free ion. This salvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance (r) in a medium of dielectric constant (e) is:

$$F = - \frac{{q}_{1} {q}_{2}}{4 {πɛ}_{0} ɛ {r}^{2}} \qquad ( Equation 2 )$$

where ε₀ is the constant of permittivity of space. The equation shows the importance of dielectric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties define a loose ion-pair.

Tight ion-pairs are formed differently from loose-ion pairs, and consequently posses different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, D. Quintanar-Guerrero et al., Pharm. Res., 14(2): 119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected.

Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity (J. Van Gelder et al., Int. J. of Pharmaceutics, 186: 127-136 (1999). The authors concluded that the formation of ion pairs may not be very efficient as a strategy to enhance trans-epithelial transport of charged hydrophilic compounds as competition by other ions found in in vivo systems may abolish the beneficial effect of counter-ions. Other authors have noted that absorption experiments with ion-pairs have not always pointed at clear-cut mechanisms (D. Quintanar-Guerrero et al., Pharm. Res., 14(2): 119-127 (1997)).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes possess bonds that are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

The lower G.I. tract absorption and bioavailablility of iron-transport moiety complexes is determined according to the procedure described in Example 2. Briefly, an animal model commonly known as the “intracolonic ligated model is used, where the complexes are intubated directly into a ligated section of the colon. Absorption of the complexes is evaluated from blood samples taken from the animal as a function of time after intubation. A rise in the hematocrit level in the blood is indicative of absorption. Comparison of the change in hematocrit level upon intubation of ferrous sulfate to the change in hematocrit level upon intubation of ferrous-laurate complex, ferrous-caprate complex, ferrous-oleate complex, and ferrous-palmitate complex shows an increased absorption of at least two-fold, preferably four-fold, more preferably eight fold, when the iron is provided in the form of an iron-transport moiety complex.

III. Exemplary Dosage Forms and Methods of Use

The complex described above provides an enhanced absorption rate in the G.I. tract, and in particular in the lower G.I. tract. Dosage forms and methods of treatment using the complex and its increased colonic absorption will now be described. It will be appreciated that the dosage forms described below are merely exemplary.

A variety of dosage forms are suitable for use with the iron-transport moiety complex. A dosage form that permits once daily dosing to achieve a therapeutic efficacy for at least about 15 hours, more preferably for at least 18 hours, and still more preferably for at least about 20 hours are provided, due the enhanced colonic absorption achieved by the complex. The dosage form may be configured and formulated according to any design that delivers a desired dose of iron. Typically, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18^{th} Ed., pp. 1682-1685 (1990).

A specific example of a dosage form suitable for use with the iron-transport moiety complex is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35: 1-21 (1995). Osmotic dosage forms are also described in detail in the following U.S. patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

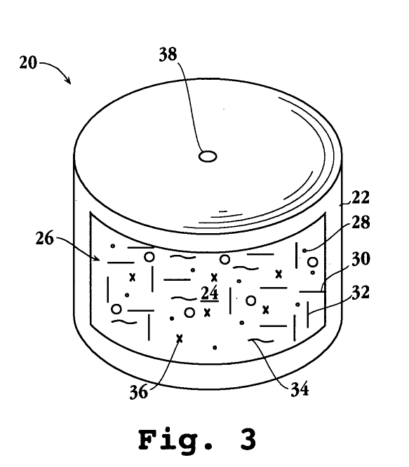

An exemplary dosage form, referred to in the art as an elementary osmotic pump dosage form, is shown in FIG. 3. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising an iron-transport moiety complex 28 in an admixture with selected excipients. The excipients are adapted to provide an osmotic activity gradient for attracting fluid from an external environment through wall 22 and for forming a deliverable iron-transport moiety complex formulation upon imbibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable iron-transport moiety complex formulation (e.g., a solution, suspension, slurry or other flowable composition) within the internal compartment. The deliverable iron-transport moiety complex formulation is released through an exit 38 as fluid continues to enter the internal compartment. Even as drug formulation is released from the dosage form, fluid continues to be drawn into the internal compartment, thereby driving continued release. In this manner, iron-transport moiety is released in a sustained and continuous manner over an extended time period.

Preparation of a dosage form like that shown in FIG. 3 is described in Example 3.

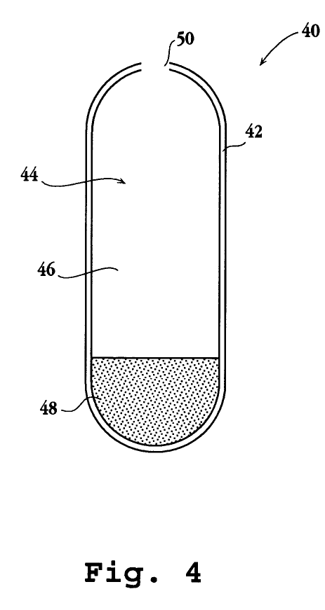

FIG. 4 is a schematic illustration of another exemplary osmotic dosage form. Dosage forms of this type are described in detail in U.S. Pat. Nos. 4,612,008; 5,082,668; and 5,091,190, which are incorporated by reference herein. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 4, or can have one or more intervening layers separating the push layer and drug layer.

Drug layer 46 comprises an iron-transport moiety complex in an admixture with selected excipients, such as those discussed above with reference to FIG. 3. An exemplary dosage form can have a drug layer was comprised of ferrous-laurate complex, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIG. 4, the dosage form can optionally include an overcoat (not shown) for color coding the dosage forms according to dose or for providing an immediate release of iron or another mineral, vitamin, or drug.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling drug from the drug layer throughout the period during which the dosage form is in the gastrointestinal tract. In this way, the dosage form provides a continuous supply of iron-transport moiety complex to the gastrointestinal tract for a period of 15 to 20 hours, or through substantially the entire period of the dosage form's passage through the G.I. tract. Since the iron-transport moiety complex is readily absorbed in both the upper and lower G.I. tracts administration of the dosage form provides delivery of iron into the blood stream over the 15-20 hour period of dosage form transit in the G.I. tract.

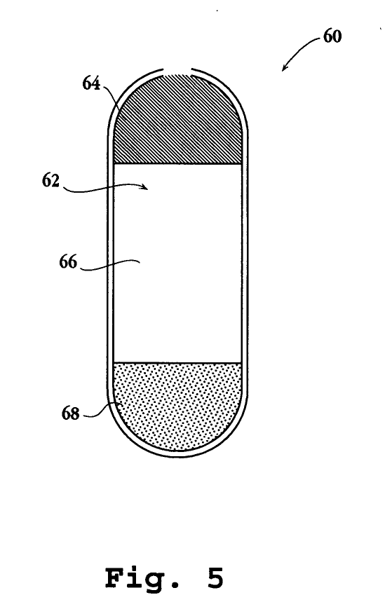

Another exemplary dosage form is shown in FIG. 5. Osmotic dosage form 60 has a tri-layered core 62 comprised of a first layer 64 of an iron salt, such as ferrous sulfate, a second layer 66 of an iron-transport moiety complex, and a third layer 68 referred to as a push layer. Dosage forms of this type are described in detail in U.S. Pat. Nos. 5,545,413; 5,858,407; 6,368,626, and 5,236,689, which are incorporated by reference herein. As set forth in Example 4, tri-layered dosage forms are prepared to have a first layer of 85.0 wt % ferrous sulfate, 10.0 wt % polyethylene oxide of 100,000 molecular weight, 4.5 wt % polyvinylpyrrolidone having a molecular weight of about 35,000 to 40,000, and 0.5 wt % magnesium stearate. The second layer is comprised 93.0 wt % ferrous-iron complex (prepared as described in Example 1), 5.0 wt % polyethylene oxide 5,000,000 molecular weight, 1.0 wt % polyvinylpyrrolidone having molecular weight of about 35,000 to 40,000, and 1.0 wt % magnesium stearate.

The push layer consists of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate. The semi-permeable wall is comprised of 80.0 wt % cellulose acetate having a 39.8% acetyl content and 20.0% polyoxyethylene-polyoxypropylene copolymer.

The dissolution rate of the dosage forms shown in FIGS. 3-5 are determined according to procedure set forth in Example 5. Depending on the dosage form, for example a one-layer dosage form (e.g, FIG. 3), release of iron-transport moiety complex begins after contact with an aqueous environment. In the dosage form illustrated in FIG. 5, release of ferrous sulfate, present in the drug layer adjacent the exit orifice, is released initially. About 8 hours after contact with an aqueous environment, release of ferrous-laurate complex occurs, and continues at a substantially constant rate for 8 hours longer. It will be appreciated that this dosage form is designed to release ferrous sulfate while in transit in the upper G.I. tract, corresponding approximately to the first eight hours of transit. Ferrous-laurate complex is released as the dosage form travels through the lower G.I. tract, approximately corresponding to times longer than about 8 hours after ingestion. This design takes advantage of the increased colonic absorption provided by the complex.

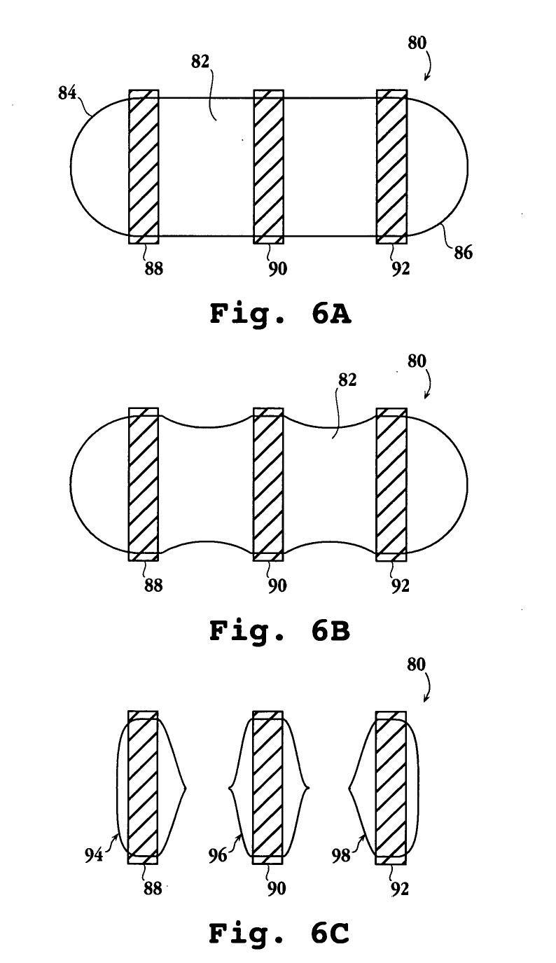

FIGS. 6A-6C illustrate another exemplary dosage form, known in the art and described in U.S. Pat. Nos. 5,534,263; 5,667,804; and 6,020,000, which are specifically incorporated by reference herein. Briefly, a cross-sectional view of a dosage form 80 is shown prior to ingestion into the gastrointestinal tract in FIG. 6A. The dosage form is comprised of a cylindrically shaped matrix 82 comprising an iron-transport moiety complex. Ends 82, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and in Example 6 below.

After ingestion of dosage form 80, regions of matrix 82 between bands 88, 90, 92 begin to erode, as illustrated in FIG. 6B. Erosion of the matrix initiates release of the iron-transport moiety complex into the fluidic environment of the G.I. tract. As the dosage form continues transit through the G.I. tract, the matrix continues to erode, as illustrated in FIG. 6C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

It will be appreciated that the dosage forms described in FIGS. 3-6 are merely exemplary of a variety of dosage forms designed for and capable of achieving delivery of an iron-transport moiety complex to the G.I. tract. Those of skill in the pharmaceutical arts can identify other dosage forms that would be suitable.

In another aspect, the invention provides a method for treating an iron-deficiency in a patient by administering a composition or a dosage form that contains a complex of iron and a transport moiety, the complex characterized by a tight-ion pair bond between the iron and the transport moiety. A composition comprising the complex and a pharmaceutically-acceptable vehicle are administered to the patient, typically via oral administration.

The dose administered is generally adjusted in accord with the age, weight, and condition of the patient, taking into consideration the dosage form and the desired result. In general, the dosage forms and compositions of the iron-transport moiety complex are administered in amounts recommended for iron therapy, as set forth in the Physician's Desk Reference. Because of the enhanced absorption provided by the complex, the dose will be lower than that typically recommended for oral therapies with ferrous sulfate (FEOSOL), ferrous fumarate (FEOSTAT), and ferrous gluconate (FEROGON). The average dose for treatment of iron-deficiency using these conventional oral therapies is about 2-3 mg/kg of iron per day, or about 200 mg/day. For prevention of iron deficiency, such as in pregnant women, a dose of 15-30 mg/day is recommended. Administration of iron in the form of the complex will decrease the required dose by at least one half, preferably by at least two-fold, due to the improved absorption. In one embodiment, an dosage form that provides a daily iron dose of between 2-20 mg is provided, where the iron is provided in the form of an iron-transport moiety complex.

From the foregoing, it can be seen how various objects and features of the invention are met. A complex comprised of iron and a transport moiety, the iron and transport moiety associated by a non-covalent tight-ion pair bond, provides an enhanced colonic absorption of iron, relative to that observed for ferrous sulfate administered orally. The complex is prepared from a novel process, where iron is contacted with a transport moiety, such as a fatty acid, solubilized in an solvent, the solvent being less polar than water, the lower polarity evidenced, for example, by a lower dielectric constant. Contact of iron with the transport moiety-solvent mixture results in formation of a complex between iron and the transport moiety, where the two species are associated by a bond that is not an ionic bond and that is not a covalent bond, but is a tight-ion pair bond.

Improved G.I tract absorption of iron is provided by the use of a complex of a transport moiety and iron. Dosage forms enable the release in the upper G.I. tract of iron-transport moiety complexes, for absorption in the upper G.I. tract, and the release of iron-transport moiety complexes in the lower G.I. tract for improved absorption therein. These dosage forms provide for absorption by the body of iron through a period of 10-24 hours, alternatively 12-20 hours, thus enabling a true once-daily dosage form for iron.

### IV. EXAMPLES

The following examples further illustrate the invention described herein and are in no way intended to limit the scope of the invention.

### Example 1

### Preparation of Iron-Fatty Acid Complex

The following steps are carried out to form a ferrous-fatty acid complex. The reaction is illustrated in FIG. 2C.
1. 9.15 grams of FeSO₄O₄.7H₂O were dissolved into 300 mL methanol in a beaker.
2. 14.64 grams of lauric acid sodium (sodium laurate) were dissolved into 300 mL methanol in a second beaker.
3. The solution of step 1 was added dropwise to the solution of step 2. The mixture was stirred for 1˜5 h at room temperature to produce a precipitate of Na₂SO₄. The solution was stirred overnight.
4. The precipitate from step 3 was removed via vacuum filtration using with #42 Whatman filter paper; the filtrate was captured in a funnel. The precipitate washed three times with methanol; the filtrate was captured into the funnel.
5. The filtrate solution of step 4 was placed in a crystallizing dish and placed in a hood to evaporate the solvent. A beige precipitate formed. The precipitate was placed on a vacuum filter and the remaining solvent was removed via vacuum filtration. The filter cake was placed in a crystallizing dish and placed in a vacuum oven overnight to dry.

The melting point of the precipitate was determined to be between 38-38° C.

### Example 2

### In Vivo Bioavailability of Iron-Transport Moiety Complex

The lower G.I. absorption and bioavailability of iron-transport moiety complexes is evaluated using an animal model commonly known as the “intracolonic ligated model”. Surgical preparation of a fasted anesthetized 0.3-0.5 kg Sprague-Dawley male rats proceeds as follows. A segment of proximal colon is isolated and the colon is flushed of fecal materials. The segment is ligated at both ends while a catheter is placed in the lumen and exteriorized above the skin for delivery of test formulations. The colonic contents are flushed out and the colon is returned to the abdomen of the animal. Depending on the experimental set up, the test formulation is added after the segment is filled with 1 mL/kg of 20 mM sodium phosphate buffer, pH 7.4, to more accurately simulate the actual colon environment in a clinical situation.

Rats are allowed to equilibrate for approximately 1 hour after surgical preparation and prior to exposure to each iron-transport moiety complex. The test compounds are administered as an intracolonic bolus and delivered at 10 mg iron (as Fe⁺²/rat). Blood samples are obtained from the jugular catheter were at 0, 15, 30, 60, 90, 120, 180 and 240 minutes and are analyzed for blood iron concentration. At the end of the 4 hour test period, the rats are euthanized with an overdose of pentobarbital. Colonic segments from each rat are excised and opened longitudinally along the anti-mesenteric border. Each segment is observed macroscopically for irritation and any abnormality noted. The excised colons are placed on graph paper and measured to approximate colonic surface area.

The above procedure is used to evaluate the absorption of ferrous sulfate salt, and of ferrous-laurate complex, ferrous-caprate complex, ferrous-oleate complex, and ferrous-palmitate complex.

### Example 3

### Preparation of Dosage Form Comprising an Iron-Transport Moiety Complex

A device as shown in FIG. 3 is prepared as follows. A compartment forming composition comprising, in weight percent, 92.25% iron-transport moiety complex, 5% potassium carboxypolymethylene, 2% polyethylene oxide having a molecular weight of about 5,000,000, and 0.5% silicon dioxide are mixed together. Next, the mixture is passed through a 40 mesh stainless steel screen and then dry blended in a V-blender for 30 minutes to produce a uniform blend. Next, 0.25% magnesium stearate is passed through an 80 mesh stainless steel screen, and the blend given an additional 5 to 8 minutes blend. Then, the homogeneously dry blended powder is placed into a hopper and fed to a compartment forming press, and known amounts of the blend compressed into 5/8 inch oval shapes designed for oral use. The oval shaped precompartments are coated next in an Accela-Cota® wall forming coater with a wall forming composition comprising 91% cellulose acetate having an acetyl content of 39.8% and 9% polyethylene glycol 3350. After coating, the wall coated drug compartments are removed from the coater and transferred to a drying oven for removing the residual organic solvent used during the wall forming procedure. Next, the coated devices are transferred to a 50° C. forced air oven for drying about 12 hours. Then, a passageway is formed in the wall of the device using a laser.

### Example 4

### Preparation of Dosage Form Comprising an Iron-Transport Moiety Complex

A dosage form comprising a layer of ferrous sulfate and a layer of ferrous-laurate complex, as illustrated in FIG. 5, is prepared as follows.

10 grams of ferrous sulfate, 1.18 g of polyethylene oxide of 100,000 molecular weight, and 0.53 g of polyvinylpyrrolidone having molecular weight of about 38,000 are dry blended in a conventional blender for 20 minutes to yield a homogenous blend. Next, 4 mL denatured anhydrous alcohol is added slowly, with the mixer continuously blending, to the three component dry blend. The mixing is continued for another 5 to 8 minutes. The blended wet composition is passed through a 16 mesh screen and dried overnight at room temperature. Then, the dry granules are passed through a 16 mesh screen and 0.06 g of magnesium stearate are added and all the ingredients are dry blended for 5 minutes. The fresh granules are ready for formulation as the initial dosage layer in the dosage form.

The layer containing ferrous-laurate complex in the dosage form is prepared as follows. First, 9.30 grams of ferrous-laurate complex, prepared as described in Example 1, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients were dry blended for 5 minutes.

A push layer comprised of an osmopolymer hydrogel composition is prepared as follows. First, 58.67 g of pharmaceutically acceptable polyethylene oxide comprising a 7,000,000 molecular weight, 5 g Carbopol® 974P, 30 g sodium chloride and 1 g ferric oxide were separately screened through a 40 mesh screen. The screened ingredients were mixed with 5 g of hydroxypropylmethylcellulose of 9,200 molecular weight to produce a homogenous blend. Next, 50 mL of denatured anhydrous alcohol was added slowly to the blend with continuous mixing for 5 minutes. Then, 0.080 g of butylated hydroxytoluene was added followed by more blending. The freshly prepared granulation was passed through a 20 mesh screen and allowed to dry for 20 hours at room temperature (ambient). The dried ingredients were passed through a 20 mesh screen and 0.25 g of magnesium stearate was added and all the ingredients were blended for 5 minutes.

The tri-layer dosage form is prepared as follows. First, 118 mg of the ferrous sulfate composition is added to a punch and die set and tamped, then 598 mg of the ferrous-laurate composition is added to the die set as the second layer and again tamped. Then, 358 mg of the hydrogel composition is added and the three layers are compressed under a compression force of 1.0 ton (1000 kg) into a 9/32 inch (0.714 cm) diameter punch die set, forming an intimate tri-layered core (tablet).

A semipermeable wall-forming composition is prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. The wall-forming composition is sprayed onto and around the tri-layerd core to provide a 60 to 80 mg thickness semi-permeable wall.

Next, a 40 mil (1.02 mm) exit orifice is laser drilled in the semipermeable walled tri-layered tablet to provide contact of the ferrous-sulfate layer with the exterior of the delivery device. The dosage form is dried to remove any residual solvent and water.

### Example 5

### In Vitro Dissolution of a Dosage Form Containing an Iron-Transport Moiety Complex

The in vitro dissolution rates of dosage forms prepared as described in Examples 3 and 4 are determined by placing a dosage form in metal coil sample holders attached to a USP Type VII bath indexer in a constant temperature water bath at 37° C. Aliquots of the release media are injected into a chromatographic system to quantify the amounts of iron released into a medium simulating artificial gastric fluid (AGF) during each testing interval.

### Example 6

### Preparation of Dosage Form Comprising an Iron-Transport Moiety Complex

A dosage form as illustrated in FIGS. 6A-6C is prepared as follows. A unit dose for prolonged release of the ferrous-laurate complex is prepared as follows. The desired dose of iron in the form of ferrous-laurate complex is passed through a sizing screen having 40 wires per inch. 20 grams of a hydroxypropyl methylcellulose having a hydroxypropyl content of 8 wt %, a methoxyl content of 22 wt %, and a number average molecular weight of 27,800 grams per mole are passed through a sizing screen with 100 wires per inch. The sized powders are tumble mixed for 5 minutes. Anhydrous ethanol is added to the mixture with stirring until a damp mass is formed. The damp mass is passed through a sizing screen with 20 wires per inch. The resulting damp granules are air dried overnight, and then passed again through the 20 mesh sieve. 2 grams of the tabletting lubricant, magnesium stearate, are passed through a sizing screen with 80 wires per inch. The sized magnesium stearate is blended into the dried granules to form the final granulation.

905 mg portions of the final granulation are placed in die cavities having inside diameters of 0.281 inch. The portions are compressed with deep concave punches under a pressure head of 1 ton, forming longitudinal capsule-shaped tablets.

The capsules are fed into a Tait Capsealer Machine (Tait Design and Machine Co., Manheim, Pa.) where three bands are printed onto each capsule. The material forming the bands is a mixture of 50 wt % ethylcellulose dispersion (Surelease®, Colorcon, West Point, Pa.) and 50 wt % ethyl acrylate methylmethacrylate (Eudragit® NE 30D, Rohm Pharma, Weiterstadt, Germany). The bands are applied as an aqueous dispersion and the excess water is driven off in a current of warm air. The diameter of the bands is 2 millimeters.

1. A substance comprised of iron and a transport moiety, the iron and the transport moiety forming a complex.

2. The compound of claim 1, wherein said transport moiety is a fatty acid of the form CH₃(CₙH₂ₙ)COOH, where n is from 2-16.

3. The compound of claim 2, wherein said fatty acid is capric acid or lauric acid.

4. A composition, comprising,
a complex consisting essentially of iron and a transport moiety, and
a pharmaceutically acceptable vehicle,
wherein said complex has an absorption in the lower gastrointestinal tract at least 2 fold higher than ferrous sulfate.

5. The composition of claim 4, wherein said transport moiety is a fatty acid of the form CH₃(CₙH₂ₙ)COOH, where n is from 2-16.

6. The composition of claim 5, wherein said fatty acid is capric acid or lauric acid.

7. A dosage form comprising the composition of claim 4.

8. A dosage form comprising the complex of claim 1.

9. The dosage form of claim 8, wherein the dosage form is an osmotic dosage form.

10. The dosage form of claim 9, comprised of (i) a push layer; (ii) drug layer comprising an iron-transport moiety complex; (iii) a semipermeable wall provided around the push layer and the drug layer; and (iv) an exit.

11. The dosage form of claim 9, comprised of (i) a semipermeable wall provided around an osmotic formulation comprising an iron-transport moiety complex, an osmagent, and an osmopolymer; and (ii) an exit.

12. The dosage form of claim 9, wherein the dosage form provides a total iron daily dose of between 20-400 mg.

13. The dosage form of claim 1, wherein the dosage form provides a total iron daily dose of between 20-400 mg.

14. An improvement in a dosage form comprising iron, the improvement comprising a dosage form comprising a complex comprised of iron and a transport moiety.

15. The improved dosage form of claim 14, wherein said transport moiety is a fatty acid of the form CH₃(CₙH₂ₙ)COOH, where n is from 2-16.

16. The improved dosage form of claim 15, wherein said fatty acid is capric acid or lauric acid.

17. A method for treating an iron-deficiency in a subject, comprising:
administering the composition of claim 4.

18. The method of claim 17, wherein said administering is via oral administration.

19. A method of preparing an iron-transport moiety complex, comprising
providing iron;
providing a transport moiety;
combining the iron and the transport moiety in the presence of a solvent having a dielectric constant less than that of water;
whereby said combining results in formation of a complex between the iron and the transport moiety.

20. The method of claim 19, wherein said combining comprises contacting in a solvent having a dielectric constant at least two fold lower than the dielectric constant of water.

21. The method of claim 20, wherein said solvent is selected from the group consisting of methanol, ethanol, acetone, benzene, methylene chloride, and carbon tetrachloride.

22. A method of improving G.I. absorption of iron, comprising
providing a complex comprising iron and a transport moiety, and
administering the complex to a patient.

23. The method of claim 22, wherein the improved absorption comprises improved absorption in the lower gastrointestinal tract.

24. The method of claim 22, wherein the improved absorption comprises improved absorption in the upper gastrointestinal tract.

25. The method of claim 22, wherein said providing includes providing a complex of iron and a fatty acid transport moiety having the form CH₃(CₙH₂ₙ)COOH, where n is from 4-16.

26. The method of claim 25, wherein said fatty acid is capric acid or lauric acid.

27. The method of claim 22, wherein said administering comprises orally administering the complex.

28. The method of claim 27, wherein said oral administration is achieved by orally administering the complex in an osmotic dosage form.

29. The method of claim 28, comprised of (i) a push layer; (ii) drug layer comprising an iron-fatty acid complex; (iii) a semipermeable wall provided around the push layer and the drug layer; and (iv) an exit.

30. The method of claim 28, comprised of (i) a semipermeable wall provided around an osmotic formulation comprising an iron-fatty acid complex, an osmagent, and an osmopolymer; and (ii) an exit.

31. The method of claim 27, wherein the dosage form provides a total daily dose of between 20-400 mg.

32. A substance comprising iron and a transport moiety, said substance prepared by a process of (i) providing iron; (ii) providing a transport moiety; (iii) combining the iron and the transport moiety in the presence of a solvent having a dielectric constant less than that of water, where said combining forms a complex between iron and the transport moiety.

33. The substance according to claim 32, wherein said transport moiety is a fatty acid of the form CH₃(CₙH₂ₙ)COOH, where n is from 4-16.

34. The substance of claim 33, wherein said fatty acid is capric acid or lauric acid.
